(19) United States
(12) Patent Application Publication
(10) Pub. No.: US 2010/0240896 A1
(43) Pub. Date: Sep. 23, 2010

(54) METHOD FOR HYDROFORMYLATION

(75) Inventors: Jens Rudolph, Worms (DE); Joachim Schmidt-Leithoff, Mannheim (DE); Rocco Paciello, Bad Duerkheim (DE); Bernhard Breit, Gundelfingen (DE); Tomas Smejkal, Freiburg (DE)

Correspondence Address: CONNOLLY BOVE LODGE & HUTZ, LLP, P O BOX 2207, WILMINGTON, DE, 19899, US

(73) Assignee: BASF SE, Ludwigshafen (DE)

(21) Appl. No.: 12/741,296

(22) PCT Filed: Nov. 4, 2008
(86) PCT No.: PCT/EP2008/064922

§ 371 Date: May 4, 2010

(30) Foreign Application Priority Data
Nov. 5, 2007 (DE) 10 2007 052 640.9

(51) Int. Cl. C07C 51/373; C07F 9/28; C07D 213/04; C07F 15/00; C07F 15/06

(52) U.S. Cl. 546/6; 562/577; 546/21; 556/21

(57) ABSTRACT

The present invention relates to a process for the hydroformylation of compounds of the formula (I),

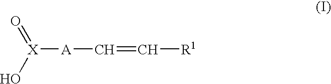

where X is C, P(R^{x}), P(O—R^{x}) S or S(═O), where R^{x} is H, alkyl, cycloalkyl, heterocycloalkyl, aryl or hetaryl; A is a divalent bridging group having from 1 to 4 bridging atoms; and R¹ is H, alkyl, alkenyl, alkynyl, cycloalkyl, heterocycloalkyl, aryl or hetaryl; or salts thereof;
in which the compound of the formula (I) is reacted with carbon monoxide and hydrogen in the presence of a catalyst comprising a complex of a metal of transition group VIII with a compound of the formula (II),

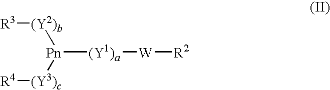

where Pn is a pnicogen atom; W is a divalent bridging group having from 1 to 8 bridging atoms; R² is a functional group capable of forming an intermolecular, noncovalent bond with the group —X(═O)OH; R³, R⁴ are each alkyl, cycloalkyl, heterocycloalkyl, aryl or hetaryl; a, b, c are each 0 or 1; and Y¹, Y² and Y³ are each O, S, NR^{a} or SiR^{b}R^{c};
and also compounds of the formula (II.a),

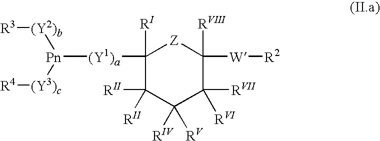

where W′ is a divalent bridging group having from 1 to 5 bridging atoms between the flanking bonds, Z is O, S, S(═O), S(═O)₂, N(R^{IX}) or C(R^{IX})(R^{X}); and R^{I} to R^{X} are each, independently of one another, H, halogen, nitro, cyano, amino, alkyl, etc.; or two radicals R^{I}, R^{II}, R^{IV}, R^{VI}, R^{VIII} and R^{IX} together represent the second part of a double bond.

The present invention relates to a process for the hydroformylation of unsaturated compounds which have a functional group capable of forming an intermolecular, noncovalent bond, in which this compound is reacted with carbon monoxide and hydrogen in the presence of a catalyst comprising a complex of a metal of transition group VIII with a pnicogen-comprising compound as ligand, where the pnicogen-comprising compound has a functional group which is complementary to the functional group capable of forming an intermolecular, noncovalent bond of the compound to be hydroformylated, such ligands, catalysts and their use.

Hydroformylation or the oxo process is an important industrial process and is employed for preparing aldehydes from unsaturated compounds, carbon monoxide and hydrogen. These aldehydes can, if appropriate, be hydrogenated by means of hydrogen in the same process to give the corresponding oxo alcohols. The reaction itself is strongly exothermic and generally proceeds under superatmospheric pressure and at elevated temperatures in the presence of catalysts. Catalysts used are Co, Rh, Ir, Ru, Pd or Pt compounds or complexes which may be modified by means of N- or P-comprising ligands to influence the activity and/or selectivity.

In the hydroformylation reaction of unsaturated compounds having more than two carbon atoms, the formation of mixtures of isomeric aldehydes can occur due to the possible CO addition onto each of the two carbon atoms of a double bond. In addition, double bond isomerization, i.e. a shift of internal double bonds to a terminal position and vice versa, can also occur when using unsaturated compounds having at least four carbon atoms. Furthermore, complex and difficult-to-separate product mixtures can be obtained from the hydroformylation when mixtures of unsaturated compounds are used.

The use of pnicogen-comprising and in particular phosphorus-comprising ligands for stabilizing and/or activating the catalyst metal in rhodium-catalyzed low-pressure hydroformylation is known. Suitable phosphorus-comprising ligands are, for example, phosphines, phosphinites, phosphonites, phosphites, phosphoramidites, phospholes and phosphabenzenes. The most widespread ligands at present are triarylphosphines, e.g. triphenylphosphine and sulfonated triphenylphosphine, since these have sufficient stability under the reaction conditions. However, a disadvantage of these ligands is that generally only very large excesses of ligand give satisfactory yields.

A publication by B. Breit and W. Seiche in J. Am. Chem. Soc. 2003, 125, 6608-6609, describes the dimerization of monodentate ligands via hydrogen bonds to form bidentate donor ligands and their use in hydroformylation catalysts having high regioselectivity.

EP 1 486 481 describes a process for the hydroformylation of olefins in the presence of a catalyst comprising at least one complex of a metal of transition group VIII with monophosphorus compounds capable of dimerization via noncovalent bonds as ligands.

DE 10 2006 041 064 describes phosphorus compounds comprising peptide groups,

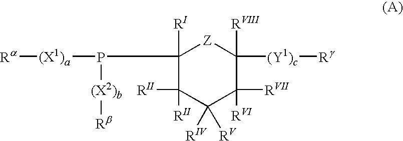

where Y¹ is a divalent bridging group having one bridging atom between the flanking bonds, R^{α} and R^{β} are each alkyl, cycloalkyl, heterocycloalkyl, aryl or hetaryl or together with the phosphorus atom and, if present, the groups X¹ and X² to which they are bound form a 5- to 8-membered heterocycle, R^{γ} is a peptide group comprising at least two amino acid units, X¹ and X² are selected from among O, S, SiR^{ε}R^{ξ} and NR^{η}, Z is NR^{IX} or CR^{IX}R^{X}, R^{I} to R^{X} are each hydrogen, alkyl, cycloalkyl, heterocycloalkyl, aryl, hetaryl, etc., where two adjacent radicals R^{I}, R^{II}, R^{IV}, R^{VI}, R^{VIII} and R^{IX} can together also represent the second bond of a double bond between the ring atoms, and a, b and c are each 0 or 1; catalysts comprising such a compound as ligand and also a process for carrying out hydroformylation in the presence of such a catalyst.

PCT/EP 2007/059722 (WO 2008/031889) describes catalysts comprising at least one metal complex having at least two pnicogen-comprising compounds capable of dimerization via ionic interactions as ligands, where the ligands have functional groups which are complementary to one another or two ligands having two noncomplementary functional groups and additionally a multivalent ionic and/or ionogenic compound complementary to the functional groups of the two ligands are used, and also processes in which such catalysts are employed.

None of the abovementioned documents describes making the ligands capable of aggregating with the compound to be reacted (substrate).

It is an object of the present invention to provide a hydroformylation process which is suitable for the chemoselective and regioselective hydroformylation of unsaturated compounds which comprise a functional group capable of forming intermolecular, noncovalent bonds. In this process, hydroformylation catalysts which display not only a high selectivity in respect of the substrate but also a high regioselectivity and/or a high selectivity in favor of hydroformylation over hydrogenation and/or make a high space-time yield possible should preferably be used in the process.

It has now surprisingly been found that this object is achieved by the use of monopnicogen ligands which are capable of forming intermolecular, noncovalent bonds with the compound to be reacted (substrate). In this way, a high regioselectivity of the hydroformylation reaction and a high selectivity in respect of the substrate reacted or the functional group reacted are achieved. The compounds according to the invention are therefore particularly advantageous for the selective hydroformylation of mixtures of unsaturated compounds or the selective hydroformylation of unsaturated compounds which have more than one functional group capable of reacting.

The present invention therefore provides a process for the hydroformylation of compounds of the formula (I),

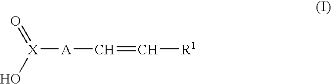

where
X is C, P(R^{x}), P(O—R^{x}), S or S(═O), where R^{x} is H, alkyl, cycloalkyl, heterocycloalkyl, aryl or hetaryl, where alkyl may have 1, 2, 3, 4 or 5 substituents selected from among halogen, cyano, nitro, alkoxy, cycloalkyl, cycloalkoxy, heterocycloalkyl, heterocycloalkoxy, aryl, aryloxy, hetaryl and hetaryloxy and where cycloalkyl, heterocycloalkyl, aryl and hetaryl may have 1, 2, 3, 4 or 5 substituents selected from among alkyl and the substituents mentioned above for alkyl,
A is a divalent bridging group having from 1 to 4 bridging atoms between the flanking bonds and
R¹ is H, alkyl, alkenyl, alkynyl, cycloalkyl, heterocycloalkyl, aryl or hetaryl, where alkyl, alkenyl and alkynyl may have 1, 2, 3, 4 or 5 substituents selected from among halogen, cyano, nitro, alkoxy, cycloalkyl, cycloalkoxy, heterocycloalkyl, heterocycloalkoxy, aryl, aryloxy, hetaryl and hetaryloxy and where cycloalkyl, heterocycloalkyl, acyl and hetaryl may have 1, 2, 3, 4 or 5 substituents selected from among alkyl and the substituents mentioned above for alkyl, alkenyl and alkynyl,
   or salts thereof,
   in which the compound of the formula (I) is reacted with carbon monoxide and hydrogen in the presence of a catalyst comprising at least one complex of a metal of transition group VIII of the Periodic Table of the Elements with at least one compound of the formula (II),

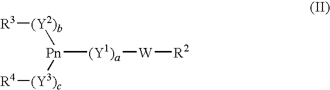

where
Pn is a pnicogen atom;
W is a divalent bridging group having from 1 to 8 bridging atoms between the flanking bonds,
R² is a functional group capable of forming at least one intermolecular, noncovalent bond with the —X(═O)OH group of the compound of the formula (I),
R³ and R⁴ are each, independently of one another, alkyl, cycloalkyl, heterocycloalkyl, aryl or hetaryl, where alkyl may have 1, 2, 3, 4 or 5 substituents selected from among halogen, cyano, nitro, alkoxy, cycloalkyl, cycloalkoxy, heterocycloalkyl, heterocycloalkoxy, aryl, aryloxy, hetaryl and hetaryloxy and where cycloalkyl, heterocycloalkyl, aryl and hetaryl may have 1, 2, 3, 4 or 5 substituents selected from among alkyl and the substituents mentioned above for alkyl; or
   together with the pnicogen atom and, if present, the radicals Y² and Y³ form a 5- to 8-membered heterocycle which may additionally be fused with one, two, three or four cycloalkyl, heterocycloalkyl, aryl or hetaryl groups, where the heterocycle and, if present, the fused-on groups each have, independently of one another, 1, 2, 3, 4 or 5 substituents selected from among halogen, cyano, nitro, alkyl, alkoxy, cycloalkyl, cycloalkoxy, heterocycloalkyl, heterocycloalkoxy, acyl, aryloxy, hetaryl and hetaryloxy,
a, b and c are each, independently of one another, 0 or 1 and
Y¹, Y² and Y³ are each, independently of one another, O, S, NR^{a} or SiR^{b}R^{c}, where R^{a}, R^{b} and R^{c} are each, independently of one another, hydrogen, alkyl, cycloalkyl, heterocycloalkyl, aryl or hetaryl, where alkyl may have 1, 2, 3, 4 or 5 substituents selected from among halogen, cyano, nitro, alkoxy, cycloalkyl, cycloalkoxy, heterocycloalkyl, heterocycloalkoxy, aryl, aryloxy, hetaryl and hetaryloxy and where cycloalkyl, heterocycloalkyl, aryl and hetaryl may, if appropriate, have 1, 2, 3, 4 or 5 substituents selected from among alkyl and the substituents mentioned above for alkyl.

Furthermore, the present invention provides the compounds of the formula (II.a) which according to the invention are used as ligands

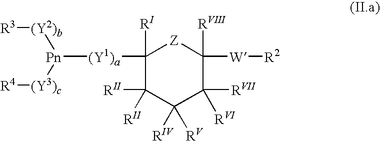

where
a, b, c, Pn, R², R³, R⁴, Y¹, Y² and Y³ each have one of the meanings given above,
W′ is a divalent bridging group having from 1 to 5 bridging atoms between the flanking bonds,
Z is N(R^{IX}) or C(R^{IX})(R^{X}) and
R^{I}, R^{II}, R^{III}, R^{IV}, R^{V}, R^{VI}, R^{VII}, R^{VIII}, R^{IX} and R^{X} are each independently of one another, H, halogen, nitro, cyano, amino, alkyl, alkoxy, alkylamino, dialkylamino, cycloalkyl, heterocycloalkyl, aryl or hetaryl,
   or two radicals R^{I}, R^{II}, R^{IV}, R^{VI}, R^{VIII} and R^{IX} bound to adjacent ring atoms together represent the second part of a double bond between the adjacent ring atoms, where a six-membered ring can have up to three noncumulated double bonds,
      catalysts comprising at least one complex of a metal of transition group VIII of the Periodic Table of the Elements with at least one compound of the formula (II.a) and also the use of such catalysts for hydroformylation.

According to the invention, ligands of the formula (II) or (II.a) which have a functional group R² capable of forming intermolecular, noncovalent bonds with the substrate of the formula (I) are used. These bonds are preferably hydrogen bonds or ionic bonds, in particular hydrogen bonds. The functional groups capable of forming intermolecular noncovalent bonds make the ligands capable of association with the substrate, i.e. capable of forming aggregates in the form of hetero-dimers.

A pair of functional groups of the ligand and of the substrate which are capable of forming intermolecular noncovalent bonds is referred to as “complementary functional groups” for the purposes of the present invention. “Complementary compounds” are ligand/substrate pairs which have functional groups which are complementary to one another. Such pairs are capable of association, i.e. capable of forming aggregates.

For the purposes of the present invention, “halogen” is fluorine, chlorine, bromine or iodine, preferably fluorine, chlorine or bromine.

For the purposes of the present invention, “pnicogen” is phosphorus, arsenic, antimony or bismuth, in particular phosphorus.

For the purposes of the present invention, the term “alkyl” refers to straight-chain and branched alkyl groups. Preference is given to straight-chain or branched C₁-C₂₀-alkyl groups, preferably C₁-C₁₂-alkyl groups, particularly preferably C₁-C₈-alkyl groups and very particularly preferably C₁-C₄-alkyl groups. Examples of alkyl groups are, in particular, methyl, ethyl, propyl, isopropyl, n-butyl, 2-butyl, sec-butyl, tert-butyl, n-pentyl, 2-pentyl, 2-methylbutyl, 3-methylbutyl, 1,2 dimethylpropyl, 1,1-dimethylpropyl, 2,2-dimethylpropyl, 1-ethylpropyl, n-hexyl, 2 hexyl, 2-methylpentyl, 3-methylpentyl, 4-methylpentyl, 1,2-dimethylbutyl, 1,3 dimethylbutyl, 2,3-dimethylbutyl, 1,1-dimethylbutyl, 2,2-dimethylbutyl, 3,3 dimethylbutyl, 1,1,2-trimethylpropyl, 1,2,2-trimethylpropyl, 1-ethylbutyl, 2 ethylbutyl, 1-ethyl-2-methylpropyl, n-heptyl, 2-heptyl, 3-heptyl, 2-ethylpentyl, 1-propylbutyl, n-octyl, 2-ethylhexyl, 2-propylheptyl, nonyl, decyl.

The expression “alkyl” also comprises substituted alkyl groups which generally have 1, 2, 3, 4 or 5 substituents, preferably 1, 2 or 3 substituents and particularly preferably 1 substituent. These are preferably selected from among halogen, cyano, nitro, alkoxy, cycloalkyl, cycloalkoxy, heterocycloalkyl, heterocycloalkoxy, aryl, aryloxy, hetaryl and hetaryloxy.

For the purposes of the present invention, the expression “cycloalkyl” refers to both unsubstituted and substituted cycloalkyl groups, preferably C₃-C₇-cycloalkyl groups such as cyclopropyl, cyclobutyl, cyclopentyl, cyclohexyl or cycloheptyl. If they are substituted, these can generally bear 1, 2, 3, 4 or 5 substituents, preferably 1, 2 or 3 substituents and particularly preferably 1 substituent. These substituents are preferably selected from among alkyl, alkoxy and halogen.

For the purposes of the present invention, the term “alkenyl” refers to both unsubstituted and substituted straight-chain and branched alkenyl groups. Preference is given to straight-chain or branched C₂-C₂₀-alkenyl groups, preferably C₂-C₁₂-alkenyl groups, particularly preferably C₁-C₄-alkenyl groups and very particularly preferably C₁-C₄-alkenyl groups.

For the purposes of the present invention, “alkynyl” refers to both unsubstituted and substituted straight-chain and branched alkynyl groups. Preference is given to straight-chain or branched C₂-C₂₀-alkynyl groups, preferably C₂-C₁₂-alkynyl groups, particularly preferably C₁-C₄-alkynyl groups and very particularly preferably C₁-C₄-alkynyl groups.

For the purposes of the present invention, the term “heterocycloalkyl” refers to saturated, cycloaliphatic groups which generally have from 4 to 7, preferably 5 or 6, ring atoms and in which 1 or 2 of the ring carbons have been replaced by heteroatoms selected from among the elements O, N, S and P and which may, if appropriate, be substituted. If they are substituted, these heterocycloaliphatic groups can bear 1, 2 or 3 substituents, preferably 1 or 2 substituents, particularly preferably 1 substituent. These substituents are preferably selected from among alkyl, halogen, cyano, nitro, alkoxy, cycloalkyl, cycloalkoxy, heterocycloalkyl, heterocycloalkoxy, aryl, aryloxy, hetaryl and hetaryloxy, with particular preference being given to alkyl radicals. Examples of such heterocycloaliphatic groups are pyrrolidinyl, piperidinyl, 2,2,6,6-tetramethylpiperidinyl, imidazolidinyl, pyrazolidinyl, oxazolidinyl, morpholidinyl, thiazolidinyl, isothiazolidinyl, isoxazolidinyl, piperazinyl, tetrahydrothiophenyl, tetrahydrofuranyl, tetrahydropyranyl, dioxanyl.

For the purposes of the present invention, the expression “aryl” refers to both unsubstituted and substituted aryl groups, preferably phenyl, tolyl, xylyl, mesityl, naphthyl, fluorenyl, anthracenyl, phenanthrenyl or naphthacenyl and particularly preferably phenyl or naphthyl. If these aryl groups are substituted, they can generally bear 1, 2, 3, 4 or 5 substituents, preferably 1, 2 or 3 substituents and particularly preferably one substituent selected from among alkyl, halogen, cyano, nitro, alkoxy, cycloalkyl, cycloalkoxy, heterocycloalkyl, heterocycloalkoxy, aryl, aryloxy, hetaryl and hetaryloxy.

For the purposes of the present invention, the expression “hetaryl” refers to unsubstituted or substituted, heterocycloaromatic groups which are preferably selected from among pyridyl, quinolinyl, acridinyl, pyridazinyl, pyrimidinyl, pyrazinyl, pyrrolyl, imidazolyl, pyrazolyl, indolyl, purinyl, indazolyl, benzotriazolyl, 1,2,3-triazolyl, 1,3,4-triazolyl and carbazolyl. If these heterocycloaromatic groups are substituted, they can generally bear 1, 2 or 3 substituents selected from among alkyl, halogen, cyano, nitro, alkoxy, cycloalkyl, cycloalkoxy, heterocycloalkyl, heterocycloalkoxy, aryl, aryloxy, hetaryl and hetaryloxy.

For the purposes of the present invention, the expression “C₁-C₄-alkylene” refers to unsubstituted or substituted methylene, 1,2-ethylene, 1,3-propylene, 1,4-butylene. If this radical is substituted, it can bear 1, 2, 3 or 4 substituents selected from among alkyl, halogen, cyano, nitro, alkoxy, cycloalkyl, cycloalkoxy, heterocycloalkyl, heterocycloalkoxy, aryl, aryloxy, hetaryl and hetaryloxy.

What has been said above with regard to the expressions “alkyl”, “cycloalkyl”, “heterocycloalkyl”, “aryl” and “hetaryl” applies analogously to the expressions “alkoxy”, “cycloalkoxy”, “heterocycloalkoxy”, “aryloxy” and “hetaryloxy”.

For the purposes of the present invention, the expression “salts of compounds of the formula (I)” refers to compounds of the formula M⁺⁻O—X(═O)-A-CH═CH—R¹, where M⁺ is a cation equivalent, i.e. a monovalent cation or the part of a polyvalent cation corresponding to a single positive charge. The cation M⁺ serves merely as counterion to neutralize negatively charged substituent groups such as the ⁻O—C(═O), ^{-O—P(R}^{x})(═O), ⁻O—P(O—R^{x})(═O) or ⁻O—S(═O)₂ group and can in principle be selected freely. Preference is therefore given to using alkali metal ions, in particular Na⁺, K⁺ and Li⁺ ions, alkaline earth metal ions, in particular Ca²⁺ or Mg²⁺ ions, or onium ions such as ammonium, monoalkylammonium, dialkylammonium, trialkylammonium, tetraalkylammonium, phosphonium, tetraalkylphosphonium or tetraarylphosphonium ions.

Without wishing to be tied to a theory, it is assumed that the catalyst comprising a metal of transition group VIII of the Periodic Table of the Elements and a compound of the formula (II) forms, owing to the presence of the group R² which is capable of forming an intermolecular, noncovalent bond, an aggregate with the compound of the formula (I) whose the C—C double bond is capable of interacting with the complexed metal of transition group VIII. Accordingly, a supramolecular, cyclic transition state could be formed as an intermediate.

The process of the invention is particularly suitable for the hydroformylation of unsaturated compounds of the formula (I) which are capable of forming a strong intermolecular, noncovalent bond. Classes of compounds which have this property are, in particular, carboxylic acids, phosphonic acids, sulfonic acids and salts thereof.

Compounds of the formula (I) in which X, A and R¹ have, independently of one another or preferably in combination, one of the meanings given below are particularly suitable for the process of the invention.

X in the compounds of the formula (I) is preferably C, S(═O) or P(O—R^{x}), where R^{x} is H or in each case optionally substituted alkyl, cycloalkyl or aryl. X is particularly preferably C. Particular preference is given to X in the compounds of the formula (I) being C, P(OH) or S(═O). Very particular preference is given to X being C.

A in the compounds of the formula (I) is preferably C₁-C₄-alkylene. A is particularly preferably C₁-C₂-alkylene and very particularly preferably methylene.

R¹ in the compounds of the formula (I) is preferably H, alkyl or alkenyl.

In a specific embodiment of the process of the invention, the compounds of the formula (I) are selected from among compounds of the formula (I.a)

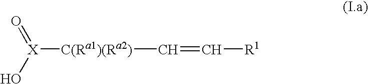

where
X is C, P(R^{x}), P(O—R^{x}), S, S(═O), where R^{x} is H or in each case optionally substituted alkyl, cycloalkyl or aryl,
R^{a1} and R^{a2} are each, independently of one another, H or C₁-C₄-alkyl and
R¹ is H, alkyl, alkenyl, alkynyl, cycloalkyl, heterocycloalkyl, aryl or hetaryl.
X in the compounds of the formula (I.a) used according to the invention is preferably C.

R^{a1} and R^{a2} in the compounds of the formula (I.a) used according to the invention is preferably H.

R¹ in the compounds of the formula (I.a) used according to the invention is preferably H or alkyl, particularly preferably H or C₁-C₈-alkyl.

Compounds of the formula (II) in which Pn, R², R³, R⁴, W, a, b, c, Y¹, Y², Y³ have, either independently of one another or preferably in combination, one of the meanings given below are particularly suitable for the process of the invention.

Pn in the compounds of the formula (II) is preferably phosphorus. Suitable examples of such compounds of the formula (II) are phosphine, phosphinite, phosphonite, phosphoramidite or phosphite compounds.

R² in the compounds of the formula (II) is a functional group comprising at least one NH group. Suitable radicals R² are —NHR^{w}, ═NH, —C(═O)NHR^{w}, C(═S)NHR^{w}, —C(═NHR^{y})NHR^{w}, —O—C(═O)NHR^{w}, —O—C(═S)NHR^{w}, —O—C(═NR^{y})NHR^{w}, —N(R^{z})—C(═O)NHR^{w}, —N(R^{z})—C(═S)NHR^{w} or —N(R^{z})—C(═NR^{y})NHR^{w}, where R^{w}, R^{y} and R^{z} are each, independently of one another, H, alkyl, cycloalkyl, aryl or hetaryl or in each case together with a further substituent of the compound of the formula (II) are part of a 4- to 8-membered ring system.

R² in the compounds of the formula (II) is particularly preferably —NH—C(═NH)NHR^{w}, where R^{w} is H, alkyl, cycloalkyl, aryl or hetaryl. R² is very particularly preferably —NH—C(═NH)NH₂.

R³ and R⁴ in the compounds of the formula (II) are preferably each optionally substituted phenyl, pyridyl or cyclohexyl. R³ and R⁴ are particularly preferably optionally substituted phenyl.

The indices a, b and c in the compounds of the formula (II) are preferably 0.

In a specific embodiment, the compounds of the formula (II) used according to the invention are selected from among compounds of the formula (II.a),

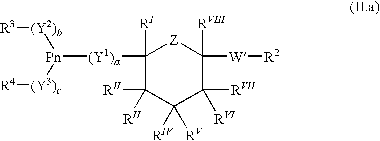

where
a, b, c, Pn, R², R³, R⁴, Y¹, Y² and Y³ have one of the meanings given above,
W′ is a divalent bridging group having from 1 to 5 bridging atoms between the flanking bonds,
Z is O, S, S(═O), S(═O)₂, N(R^{IX}) or C(R^{IX})(R^{X}) and
R^{I}, R^{II}, R^{III}, R^{IV}, R^{V}, R^{VI}, R^{VII}, R^{VIII} and, if present, R^{IX} and R^{x} are each, independently of one another, H, halogen, nitro, cyano, amino, alkyl, alkoxy, alkylamino, dialkylamino, cycloalkyl, heterocycloalkyl, aryl or hetaryl,
   or two radials R^{I}, R^{II}, R^{IV}, R^{VI}, R^{VII} and R^{IX} bound to adjacent ring atoms together represent the second part of a double bond between the adjacent ring atoms, where the six-membered ring can have up to three noncumulated double bonds.

As regards preferred meanings of a, b, c, Pn, R², R³, R⁴, Y¹, Y² and Y³, reference may be made to what has been said above with regard to the compounds of the general formula (II).

Compounds of the formula (II.a) in which a, b, c, Pn, R², R³, R⁴, R^{I}, R^{II}, R^{III}, R^{IV}, R^{V}, R^{VI}, R^{VII}, R^{VIII}, R^{IX}, R^{X}, Y¹, Y², Y³ and Z have, either independently of one another or preferably in combination, one of the meanings mentioned above as preferred or one of the following meanings are particularly suitable for the process of the invention.

W′ in the compounds of the formula (II.a) is preferably C₁-C₅-alkylene, (C₁-C₄-alkylene)carbonyl or C(═O). W′ in the compounds of the formula (II.a) is particularly preferably C(═O).

Z in the compounds of the formula (II.a) is preferably N(R^{IX}) or C(R^{IX})(R^{X}). Z is particularly preferably N(R^{IX}).

The radicals R^{I} together with R^{II}, R^{IV} together with R^{VI} and R^{VIII} together with R^{IX} in the compound of the formula (II.a) preferably in each case represent the second part of a double bond between the adjacent ring atoms, i.e. the six-membered ring in the compound of the formula (II.a) is preferably substituted benzene or pyridine.

Preference is given to the radicals R^{III}, R^{V}, R^{VII} and, if present, R^{X} in the compounds of the formula (II.a) each being, independently of one another, H, halogen, nitro, cyano, amino, C₁-C₄-alkyl, C₁-C₄-alkoxy, C₁-C₄-alkylamino or di(C₁-C₄-alkyl)amino. R^{III}, R^{V}, R^{VII} and, if present, R^{X} are particularly preferably each H.

In a particularly preferred embodiment of the process of the invention, the compounds of the formulae (II) or (II.a) are selected from among the compounds of the formulae (1) and (2)

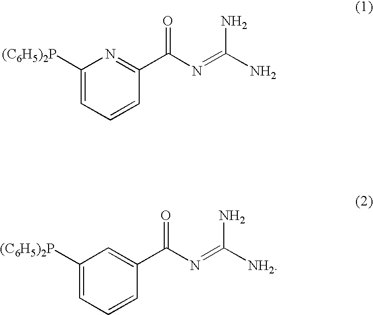

Very particular preference is given to using the compound of the formula (1) in the hydroformylation process of the invention.

The catalysts used according to the invention have at least one compound of the formula (II) or (II.a) as described above as ligand. In addition to the above-described ligands, the catalysts can additionally have at least one further ligand which is preferably selected from among halides, amines, carboxylates, acetylacetonate, arylsulfonates and alkylsulfonates, hydride, CO, olefins, dienes, cycloolefins, nitriles, N-comprising heterocycles, aromatics and heteroaromatics, ethers, PF₃, phospholes, phosphabenzenes and monodentate, bidentate and polydentate phosphine, phosphinite, phosphonite, phosphoramidite and phosphite ligands.

The catalysts used according to the invention preferably comprise at least one metal of transition group VIII of the Periodic Table of the Elements. The metal of transition group VIII is preferably Co, Ru, Rh, Ir, Pd or Pt, particularly preferably Co, Ru, Rh or Ir and very particularly preferably Rh.

In general, catalytically active species of the general formula HₓM_{y}(CO)_{z}L_{q}, where M is the metal of transition group VIII, L is a pnicogen-comprising compound of the formula (II) and q, x, y, z are integers which depend on the valence and type of the metal and the number of coordination positions occupied by the ligand L, are formed under hydroformylation conditions from the catalysts or catalyst precursors used in each case. Preference is given to z and q each being, independently of one another, at least 1, e.g. 1, 2 or 3. The sum of z and q is preferably from 1 to 5. The complexes can, if desired, additionally have at least one of the above-described further ligands.

In a preferred embodiment, the hydroformylation catalysts are prepared in situ in the reactor used for the hydroformylation reaction. However, the catalysts of the invention can, if desired, also be prepared separately and isolated by customary methods. For the in-situ preparation of the catalysts of the invention, it is possible, for example, to react at least one ligand of the formula (II) used according to the invention, a compound or a complex of a metal of transition group VIII, if appropriate at least one further additional ligand and if appropriate an activator in an inert solvent under the hydroformylation conditions.

Suitable rhodium compounds or complexes are, for example, rhodium(II) and rhodium(III) salts such as rhodium(III) chloride, rhodium(III) nitrate, rhodium(III) sulfate, potassium rhodium sulfate, rhodium(II) or rhodium(III) carboxylate, rhodium(II) and rhodium(III) acetate, rhodium(III) oxide, salts of rhodic(III) acid, trisammonium hexachlororhodate(III), etc. Rhodium complexes such as dicarbonylrhodium acetylacetonate, acetylacetonatobisethylenerhodium(I), etc., are also suitable. Preference is given to using bicarbonylrhodium acetylacetonate or rhodium acetate.

Ruthenium salts or compounds are likewise suitable. Suitable ruthenium salts are, for example, ruthenium(III) chloride, ruthenium(IV), ruthenium(VI) or ruthenium(VIII) oxide, alkali metal salts of ruthenium oxo acids, e.g. K₂RuO₄ or KRuO₄, or complexes such as RuHCl(CO)(PPh₃)₃. The metal carbonyls of ruthenium, e.g. dodecacarbonyltrisruthenium or octadecacarbonylhexaruthenium, or mixed forms in which CO has been partly replaced by ligands of the formula PR₃, e.g. Ru(CO)₃(PPh₃)₂, can also be used in the process of the invention.

Suitable cobalt compounds are, for example, cobalt(II) chloride, cobalt(II) sulfate, cobalt(II) carbonate, cobalt(II) nitrate, their amine or hydrate complexes, cobalt carboxylates such as cobalt acetate, cobalt ethylhexanoate, cobalt naphthanoate and also the cobalt caproate complex. Here too, it is possible to use the carbonyl complexes of cobalt, e.g. octacarbonyldicobalt, dodecacarbonyltetracobalt and hexadecacarbonylhexacobalt.

The abovementioned and further suitable compounds of cobalt, rhodium, ruthenium and iridium are known in principle and are adequately described in the literature or can be prepared by a person skilled in the art using methods analogous to those for the known compounds.

Suitable activators are, for example, Brönsted acids, Lewis acids such as BF₃, AlCl₃, ZnCl₂, and Lewis bases.

Suitable solvents are ethers such as tert-butyl methyl ether, diphenyl ether and tetrahydrofuran. Further possible solvents are esters of aliphatic carboxylic acids with alkanols, for example acetic esters, or oxo oils such as Palatinol™ or Texanol™, aromatics such as toluene and xylenes, hydrocarbons or mixtures of hydrocarbons.

The molar ratio of monopnicogen ligand (II) to metal of transition group VIII is generally in the range from about 1:1 to 1000:1, preferably from 2:1 to 500:1 and particularly preferably from 5:1 to 100:1.

Preference is given to a process in which the hydroformylation catalyst is prepared in situ by reacting at least one ligand (II) which can be used according to the invention, a compound or a complex of a metal of transition group VIII and, if appropriate, an activator in an inert solvent under the hydroformylation conditions.

The hydroformylation reaction can be carried out continuously, semicontinuously or batchwise.

Suitable reactors for a continuous reaction are known to those skilled in the art and are described, for example, in Ullmanns Enzyklopädie der technischen Chemie, vol. 1, 3rd edition, 1951, p. 743 et. seq.

Suitable pressure-rated reactors are likewise known to those skilled in the art and are described, for example, in Ullmanns Enzyklopädie der technischen Chemie, vol. 1, 3rd edition, 1951, p. 769 et. seq. In general, the process of the invention is carried out using an autoclave which can, if desired, be provided with a stirrer and an internal liner.

The composition of the synthesis gas comprising carbon monoxide and hydrogen which is used in the process of the invention can be varied within a wide range. The molar ratio of carbon monoxide to hydrogen is generally from about 5:95 to 70:30, preferably from about 40:60 to 60:40. Particular preference is given to using a molar ratio of carbon monoxide to hydrogen in the region of about 1:1.

The temperature in the hydroformylation reaction is generally in the range from about 20 to 180° C., preferably from about 50 to 150° C. The pressure is generally in the range from about 1 to 700 bar, preferably from 1 to 600 bar, in particular from 1 to 300 bar. The reaction pressure can be varied as a function of the activity of the inventive hydroformylation catalyst which is used. In general, the catalysts of the invention based on pnicogen-comprising compounds of the formula (II) generally make a reaction in the region of relatively low pressures, for instance in the range from 1 to 100 bar, possible.

The hydroformylation catalysts of the invention and those used according to the invention can be separated from the output from the hydroformylation reaction by customary methods known to those skilled in the art and can generally be reused for the hydroformylation.

The above-described catalysts can also be immobilized in a suitable way, e.g. by bonding via functional groups suitable as anchor groups, adsorption, grafting, etc., to a suitable support, e.g. a support composed of glass, silica gel, synthetic resins, polymers, etc. They are then suitable for use as solid-phase catalysts.

The hydroformylation activity of catalysts based on the above-described ligands of the formula (II) is generally higher than the isomerization activity in respect of the formation of internal double bonds. In the hydroformylation of unsaturated compounds which comprise a functional group capable of forming intermolecular, noncovalent bonds, the catalysts used according to the invention advantageously display high chemoselectives and regioselectives in respect of the hydroformylation of the reactive centers. Furthermore, the catalysts generally have a high stability under the hydroformylation conditions, so that they generally make it possible to achieve longer catalyst lives than when using catalysts known from the prior art. The catalysts used according to the invention advantageously also display a high activity, so that the corresponding aldehydes or alcohols are generally obtained in good yields.

The present invention further provides the compounds of the formula (II.a) used according to the invention

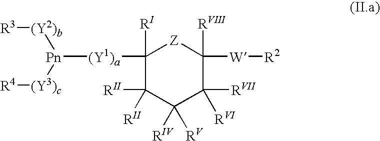

As regards the preferred meanings of the variables a, b, c, Pn, R², R³, R⁴, Y¹, Y², Y³, W′, Z, R^{I}, R^{II}, R^{III}, R^{IV}, R^{V}, R^{VI}, R^{VII}, R^{VIII} and, if present, R^{IX} and R^{X}, reference may be made to what has been said above with regard to the process of the invention.

Particular preference is given to compounds of the formula (II.a), in which Pn is phosphorus.

Preference is likewise given to compounds of the formula (II.a), in which R² is —NH—C(═NH)NHR^{w}, where R^{w} is H, alkyl, cycloalkyl, aryl or hetaryl and in particular —NH—C(═NH)NH₂.

Preference is likewise given to compounds of the formula (II.a), in which R³ and R⁴ are each optionally substituted phenyl.

Preference is likewise given to compounds of the formula (II.a), in which a, b and c are each 0.

Preference is likewise given to compounds of the formula (II.a), in which W′ is a C(═O) group.

Preference is likewise given to compounds of the formula (II.a), in which Z is N(R^{IX}).

Preference is likewise given to compounds of the formula (II.a), in which R^{I} together with R^{II}, R^{IV} together with R^{VI} and R^{VIII} together with R^{IX} are in each case the second part of a double bond between the adjacent ring atoms and R^{III}, R^{V}, R^{VII} and, if present, R^{X} are each H.

What has been said above with regard to preferred meanings of a, b, c, Pn, R², R³, R⁴, R^{I}, R^{II}, R^{III}, R^{IV}, R^{V}, R^{VI}, R^{VII}, R^{VIII}, R^{IX}, R^{X}, W′, Y¹, Y², Y³ and Z in the compounds of the formula (II.a) apply independently of one another and in particular in any combination.

The compounds of the formula (II.a) are very particularly preferably selected from among the compounds of the formulae (1) and (2)

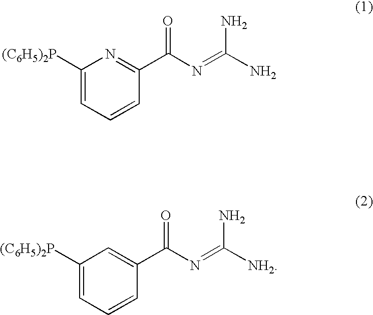

The present invention further provides the catalysts comprising at least one complex of a metal of transition group VIII of the Periodic Table of the Elements with at least one compound of the formula (II.a) as defined above which are preferably used according to the invention. As regards preferred metals of transition group VIII and preferred compounds of the formula (II.a) according to the invention, reference may be made to what has been said above.

The invention further provides for the use of catalysts comprising at least one complex of a metal of transition group VIII with at least one ligand of the formula (I) as described above for hydroformylation. As regards preferred embodiments, reference may be made to what has been said above in respect of the catalysts of the invention.

The invention is illustrated below with the aid of nonlimiting examples.

### EXAMPLES

### I. General

All reactions were carried out under an argon atmosphere in dried glass apparatuses. Air- and moisture-sensitive liquids were transferred by means of syringes. All solutions were dried and distilled using standard methods. Solutions were freed of solvents under reduced pressure using a rotary evaporator. Merck silica gel Si 60® (200-400 mesh) was used for chromatographic purification of reaction products. NMR spectra were recorded using a Varian Mercury spectrometer (300 MHz, 121 MHz and 75 MHz for ¹H, ³¹P and ¹³C), a Bruker AMX 400 (400 MHz, 162 MHz and 101 MHz for ¹H, ³¹P and ¹³C) or a Bruker DRX 500 (500 MHz, 202 MHz and 125 MHz for ¹H, ³¹P and ¹³C). As reference, TMS was used as internal standard (¹H- and ¹³C-NMR) or 85% H₃PO₄ as standard (³¹P-NMR). ¹H-NMR data are reported as follows: chemical shift (δ in ppm), multiplicity (s=singulet; bs=broad singulet; d=doublet; t=triplet; q=quartet; m=multiplet), coupling constant (Hz), integration. ¹³C-NMR data are reported as follows: chemical shift (δ in ppm), multiplicity, coupling constant (Hz). High-resolution mass spectra were recorded using a Finnigan MAT 8200. Elemental analyses were carried out using an Elementar vario (from Elementar Analysensysteme GmbH).

### II. Preparation of the Compounds of the Formula (II)

1. Preparation of N-(6-diphenylphosphanylpyridin-2-ylcarbonyl)guanidine (1)

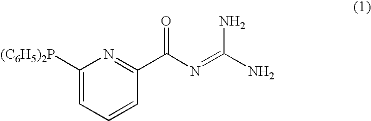

1.1 2-Bromo-6-diphenylphosphanylpyridine

n-Butyllithium (48.2 ml, 0.093 mol, 1.6 M in hexane, 1.1 eq.) was added slowly (15 min.) to a solution of 2,6-dibromopyridine (20 g, 0.084 mol, 1 eq.) in CH₂Cl₂ (750 ml) at −78° C. under argon. The reaction mixture was stirred for a further 30 minutes. Ph₂PCl (17.6 ml (95%), 0.093 mol, 1.1 eq.) was subsequently added over a period of 10 minutes and the reaction mixture obtained was stirred at −78° C. for a further 30 minutes. The solution obtained was warmed to room temperature over a period of 1.5 hours and stirred at this temperature for a further 2 hours. The reaction mixture was subsequently admixed with water (400 ml). After separation of the phases obtained, the aqueous phase was extracted with CH₂Cl₂ (2×100 ml). The organic phases were combined, dried over Na₂SO₄ and freed of the solvent under reduced pressure. The residue was dissolved in CH₂Cl₂ (100 ml) and filtered through silica gel. Evaporation of the solvent and subsequent trituration of the residue with petroleum ether/Et₂O (3:1) gave 2-bromo-6-diphenylphosphanylpyridine as a colorless solid in an amount of 26 g (yield: 90%).

M.p.: 81° C. ¹H-NMR (400.1 MHz, C₆D₆): δ=6.43-6.48 (m, 1H); 6.76-6.79 (m, 2H); 7.01-7.06 (m, 6H); 7.40-7.46 ppm (m, 4H). ¹³C{¹H}-NMR (100.6 MHz, C₆D₆): δ=126.5; 126.8 (d, J=17.3 Hz); 128.9 (d, J=7.2 Hz); 129.3; 134.6 (d, J=20.3 Hz); 136.5 (d, J=11.6 Hz); 137.6 (d, J=2.4 Hz); 143.1 (d, J=10.6 Hz); 166.6 (d, J=4.1 Hz). ³¹P {¹H}-NMR (161.97 MHz, C₆D₆): δ=−2.26. Elemental analysis [%]: calc.: C, 59.67; H, 3.83; N, 4.09; found: C, 59.47; H, 3.90; N, 4.26.

1.2 6-Diphenylphosphanylpyridin-2-ylcarboxylic acid

n-Butyllithium (40.2 ml, 0.064 mol, 1.6 M in hexane, 1.1 eq.) was added dropwise to a solution of 2-bromo-6-diphenylphosphinopyridine (20 g, 0.059 mol, 1 eq.) in CH₂Cl₂ (800 ml) at −78° C. under argon. The reaction mixture was stirred at this temperature for 75 minutes. Gaseous CO₂ was subsequently passed through the resulting solution at −78° C. for 30 minutes. The reaction mixture was heated at a temperature of −30° C. under a CO₂ atmosphere for a period of 1.5 hours. The reaction mixture was subsequently cooled back down to −78° C., saturated with CO₂ (15 minutes) and warmed to 0° C. over a period of 2 hours. The reaction mixture was extracted with aqueous hydrochloric acid (2 M, 3×200 ml). The aqueous phase was subsequently extracted with CH₂Cl₂ (2×100 ml). The organic phases were combined, dried over Na₂SO₄, filtered and freed of the solvent under reduced pressure. The yellowish, oily residue was taken up in ethyl acetate (50 ml) and filtered through a short silica gel column (washing through with ethyl acetate). Removal of the solvent and trituration of the residue with petroleum ether/diethyl ether (2:1) gave 6-diphenylphosphanylpyridin-2-ylcarboxylic acid as a slightly yellowish solid in an amount of 18 g (yield: 80%).

M.p.: 122° C. ¹H-NMR (400.1 MHz, C₆D₆): δ=6.76 (ddd, J=7.7; 7.7; 2.0 Hz, 1H); 6.96 (ddd, J=7.7; 1.8; 1.1 Hz, 1H); 7.02-7.08 (m, 6H); 7.26-7.33 (m, 4H); 7.71 (ddd, J=7.7; 1.0; 0.5 Hz, 1H); 10.66 ppm (bs, 1H). ¹³C{¹H}-NMR (100.6 MHz, C₆D₆): δ=121.9 (s); 129.0 (d, J=7.5 Hz); 129.8 (s); 131.7 (d, J=21.7 Hz); 134.5 (d, J=20.3 Hz); 135.6 (d, J=10.4 Hz); 137.6 (d, J=3.6 Hz); 147.0 (d, J=7.0 Hz); 163.1 (d, J=7.2 Hz); 163.3 ppm(s). ³¹P{¹H}-NMR (161.97 MHz, C₆D₆, H₃PO₄): δ=−2.3 ppm. Elemental analysis [%]: calc.: C, 70.36; H, 4.59; N, 4.56; found: C, 70.20; H, 4.79; N, 4.70.

1.3 N′-tert-Butoxycarbonyl-N-(6-diphenylphosphanylpyridin-2-ylcarbonyl)guanidine

1-Benzotriazolyloxytris(dimethylamino)phosphonium hexafluorophosphate (BOP, 17.6 g, 39.79 mmol, 1 eq.) was added to a solution of 6-diphenylphosphanylpyridin-2-carboxylic acid (12.23 g, 39.79 mmol, 1 eq.), N′-tert-butyloxycarbonylguanidine (9.5 g, 59.68 mmol, 1.5 eq.) and N-methylmorpholine (10.9 ml, 10.06 g, 99.5 mmol, 2.5 eq.) in DMF (250 ml) at 0° C. under an argon atmosphere. The reaction mixture was stirred at 0° C. for 2 hours and at room temperature for a further 2 hours. The reaction was monitored by means of TCL (petroleum ether/ethyl acetate/CH₃OH, 50:25:2). After addition of water (200 ml) at 0° C., the reaction product precipitated. The resulting suspension was stirred at 0° C. for 10 minutes. The white solid was subsequently isolated by filtration and washed with water (2×100 ml). The crude product was taken up in CH₂Cl₂ and filtered. The filtrate was washed with water, dried over Na₂SO₄ and freed of the solvent under reduced pressure. The residue was taken up in CH₂Cl₂/ethyl acetate (3:1) and filtered through a short silica gel column. Trituration with CH₂Cl₂/petroleum ether and subsequent removal of the solvent gave N′-tert-butoxycarbonyl-N-(6-diphenylphosphanylpyridin-2-ylcarbonyl)guanidine as a colorless solid in an amount of 14.3 g (yield: 80%). (Purification by chromatography (petroleum ether/ethyl acetate/CH₃OH, 30:10:1) is likewise possible).

Rf (SiO₂; petroleum ether/ethyl acetate/CH₃OH, 30:10:1)=0.4. M.p.: 150° C. (with decomposition). ¹H-NMR (499.7 MHz, CDCl₃): δ=1.51 (s, 9H); 7.17 (bd, J=7.8 Hz, 1H); 7.25-7.29 (m, 4H); 7.36-7.43 (m, 6H); 7.79 (td, J=7.8; 7.8; 1.3 Hz, 1H); 8.01 (d, J=7.8 Hz, 1H); 9.14 (bs, 1H); 9.25 (bs, 1H); 10.08 ppm (bs, 1H). ¹³C{¹H}-NMR (125.7 MHz, CDCl₃): δ=28.2 (s); 79.5 (s); 121.6 (s); 128.9 (d, J=7.5 Hz); 129.5 (s); 131.5 (d, J=8.6 Hz); 134.1 (d, J=19.4 Hz); 134.9 (d, J=10.7 Hz); 137.6 (s); 147.7 (d, J=14.0 Hz); 158.5 (s); 163.4 (s); 164.5 (s); 165.9 ppm (s). ³¹P{¹H}-NMR (202.3 MHz, CDCl₃): δ=−4.13 ppm. IR (Film, CH₂Cl₂): ν=3390, 2975, 1703, 1655, 1573, 1434, 1408, 1301 cm⁻¹. MS (CI): [m/e]=448.8 (100%, [M+H]⁴), 349 (45%, [M+H-Boc]⁺). Elemental analysis [%]: calc.: C, 64.28; H, 5.62; N, 12.49; found: C, 64.34; H, 5.58; N, 12.54.

1.4 N-(6-Diphenylphosphanylpyridin-2-ylcarbonyl)guanidine (1)

N′-tert-Butoxycarbonyl-N-(6-diphenylphosphanylpyridin-2-carbonyl)guanidine (10 g, 22.30 mmol, 1 eq.) and 1,3-dimethoxybenzene (3.14 ml, 3.39 g, 24.53 mmol, 1.1 eq.) were dissolved in trifluoroacetic acid (80 ml) under an argon atmosphere and stirred at room temperature for 3 hours (TLC monitoring: CH₂Cl₂/CH₃OH/triethylamine, 30:2:1, Mo—Ce reagent). The excess of trifluoroacetic acid was removed under reduced pressure. The residue was dissolved in CH₂Cl₂ (60 ml) and an Na₂CO₃ solution (20% aq., 200 ml) was added at 0° C. The two-phase mixture was stirred vigorously at 0° C. for 15 minutes. This resulted in formation of a white precipitate. The precipitate was filtered off and washed a number of times with water (as an alternative, the precipitate can be suspended in about 200 ml of water, treated with ultrasound and filtered off). The precipitate was subsequently washed again with ethyl acetate (2×25 ml), n-pentane (2×25 ml) and dried over phosphorus pentoxide under reduced pressure. N-(6-Diphenylphosphanylpyridin-2-ylcarbonyl)guanidine was obtained as dichloromethane adduct as a colorless powder in an amount of 7.55 g (yield). This compound is insoluble in customary solvents with the exception of DMSO.

Rf (SiO₂, CH₂Cl₂/CH₃OH/triethylamine-30:2:1)=0.4. M.p.: >250° C. ¹H-NMR (499.7 MHz, d⁶-DMSO): δ=6.96 (d, J=7.6 Hz, 1H); 7.25-7.34 (m, 4H); 7.38-7.44 (m, 6H); 7.76 (td, J=7.6; 7.6; 1.6 Hz, 1H); 7.94 (d, J=7.6 Hz, 1H); 6.79 and 8.05 ppm (bs, 4H). ¹³C{¹H}-NMR (125.7 MHz, d⁶-DMSO): δ=122.2 (s); 127.8 (d, J=11.8 Hz); 128.7 (d, J=6.4 Hz); 129.1 (s); 133.6 (d, J=19.3 Hz); 136.0 (d, J=11.8 Hz); 136.3 (s); 156.9 (d, J=14.0 Hz); 161.9 (d, J=7.5 Hz); 163.2 (d, J=8.6 Hz); 174.8 ppm (s). ³¹P{¹H}-NMR (161.97 MHz, d⁶-DMSO, H₃PO₄): δ=−5.63 ppm. MS (EI) [m/e]=348.0 (100%, [M]⁻¹). Elemental analysis [%]: calc. for M: C, 65.51; H, 4.92; N, 16.08; calc. for (M+0.55·CH₂Cl₂): C, 59.44; H, 4.62; N, 14.18; found: C, 59.37; H, 4.75; N, 14.49.

### 2. Preparation of N-(3-Diphenylphosphanylbenzoyl)guanidine (2)

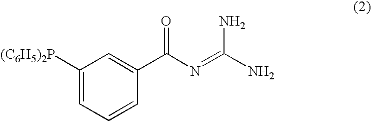

2.1 N′-tert-Butoxycarbonyl-N-(3-diphenylphosphanylbenzoyl)guanidine

1-Benzotriazolyloxytris(dimethylamino)phosphonium hexafluorophosphate (BOP, 1.733 g, 3.92 mmol, 1 eq.) was added to a solution of 3-(diphenylphosphino)benzoic acid (1.2 g, 3.92 mmol, 1 eq.), tert-butyloxycarbonylguanidine (936 mg, 5.88 mmol, 1.5 eq.) and N-methylmorpholine (862 μl, 793 mg, 7.84 mmol, 2 eq.) in dimethylformamide (DMF, 25 ml) at 0° C. The reaction mixture was stirred at 0° C. for 10 minutes and at room temperature for 4 hours under an argon atmosphere. The reaction was monitored by means of TLC (CH₂Cl₂/ethyl acetate, 10:1). After addition of water (40 ml), a precipitate foamed (stirred for 10 minutes at 0° C.). The precipitate was filtered off and washed with water (20 ml). The precipitate was dissolved in CH₂Cl₂ and filtered again. The filtrate was washed with water, dried over Na₂SO₄ and freed of the solvent under reduced pressure. The residue was purified by column chromatography (CH₂Cl₂/ethyl acetate, 10:1). Trituration with n-pentane (20 ml) at −30° C. gave N′-tert-butoxycarbonyl-N-(3-diphenylphosphanylbenzoyl)guanidine as a colorless solid in an amount of 1.195 g (yield: 68%).

Rf (SiO₂, CH₂Cl₂/ethyl acetate, 10:1)=0.65. M.p.: 79-82° C. ¹H-NMR (499.7 MHz, CDCl₃): δ=1.34 (s, 9H); 7.27-7.33 (m, 10H); 7.36-7.42 (m, 2H); 8.05 (d, J=7.0 Hz, 1H); 8.13 (d, J=7.9 Hz, 1H); 8.55 (bs, 1H); 8.56 ppm (bs, 2H). ¹³C{¹H}-NMR (125.7 MHz, CDCl₃): δ=27.9 (s); 82.9 (s); 128.3 (d, ³J=5.4 Hz); 128.6 (d, ³J=7.5 Hz); 128.7 (d); 128.8 (s); 129.5 (bs); 133.7 (d, J=19.3 Hz); 134.3 (bd, J=23.6 Hz); 136.8 (m); 137.5 (bd); 154.0 (s); 159.3 (s); 177.7 ppm (s). ³¹P{¹H}-NMR (202.3 MHz, CDCl₃, H₃PO₄): δ=−4.47 ppm. MS (EI): [m/e]=305 (85%, [M-(NHCNBoc)]⁺), 346 (100%, [M-H-Boc]⁺), 447.1 (30%, [M]⁺). Elemental analysis [%]: calc.: C, 67.1; H, 5.86 N, 9.39; found: C, 67.1; H, 5.98; N, 9.19.

2.2 N-(3-D iphenylphosphanylbenzoyl)guanidine (2)

N′-tert-Butoxycarbonyl-N-(3-diphenylphosphanylbenzoyl)guanidine (800 mg, 1.789 mmol) was dissolved in trifluoroacetic acid (8 ml) under an argon atmosphere and stirred at room temperature for 1.5 hours (TLC monitoring: CH₂Cl₂/CH₃OH/triethylamine, 30:2:1; Mo—Ce reagent). The excess of trifluoroacetic acid was removed under reduced pressure. The residue was dissolved in CH₂Cl₂ (10 ml) and extracted with an Na₂CO₃ solution (20% aq., 10 ml). The aqueous phase was extracted with CH₂Cl₂ (2×10 ml). The organic phases were combined, dried over MgSO₄, filtered and freed of the solvent under reduced pressure. The residue was triturated with n-pentane (2×10 ml) and dried under reduced pressure. N-(3-Diphenyl-phosphanylbenzoyl)guanidine was obtained as a colorless solid in an amount of 590 mg (yield: 95%).

Rf (SiO₂, acetone)=0.5. M.p.: 75-77° C. ¹H-NMR (400.1 MHz, d⁶-DMSO): δ=7.21-7.27 (m, 5H); 7.39-7.45 (m, 7H); 8.07 (d, J=7.6 Hz, 1H); 8.10 (d, J=9.1 Hz, 1H); 7.0 and 7.9 ppm (bs, 4H). ¹³C{¹H}-NMR (100.6 MHz, d⁶-DMSO, TMS): δ=128.2 (d, J=5.3 Hz); 128.7 (d, J=6.8 Hz); 128.9 (s); 129.0 (s); 133.2 (d, J=19.6 Hz); 133.6 (d, J=26.1 Hz); 135.1 (d, J=14.2 Hz); 136.2 (d, J=11.8 Hz); 136.4 (d, J=11.4 Hz); 138.5 (s); 162.1 (s); 174.3 ppm (s). ³¹P{¹H}-NMR (161.98 MHz, d⁶-DMSO): δ=−5.83 ppm. MS (EI): [m/e]=183 (25%), 330 (50%, [M-NH₃]⁺), 347 (100%, [M]⁺). High-resolution mass: calc.: 347.118750; found: 347.119402.

### II. Preparation of the Substrates

1. Preparation of (Z)-pent-3-enoic acid ((Z)-3)
1.1 (Z)-Pent-3-en-1-ol

Lindlar catalyst (45 mg) was placed in a 250 ml Schlenk flask and degassed. Quinoline (780 mg, distilled under argon), diethyl ether (150 ml, abs.) and pent-3-yn-1-ol (2.74 ml, 2.5 g, 29.7 mmol) were added. The argon atmosphere was replaced by an H₂ atmosphere. The hydrogenation was carried out at an H₂ pressure of 1 bar at room temperature for 20 hours. The reaction mixture obtained was filtered through Celite and washed through with diethyl ether. The filtrate was freed of the solvent under reduced pressure. The residue was purified by distillation (140° C./atmospheric pressure). (Z)-Pent-3-en-1-ol was obtained as a colorless liquid in an amount of 2.4 g (yield: 94%). The content of (Z)-isomer in the product obtained was >96% according to GC analysis (GC: 6890N AGILENT TECHNOLOGIES; column: 24079 SUPELCO, Supelcowax 10, 30.0 m×0.25 mm×0.25 μm; 75° C. isothermal, He flow 0.7 ml/min; (E): 18.9 min., (Z): 19.3 min.).

¹H-NMR (400.1 MHz, CDCl₃): δ=1.64 (d, J=5.6 Hz, 3H); 2.33 (pseudo-q, J=7.0 Hz, 2H); 3.63 (t, J=6.6 Hz, 2H); 5.35-5.45 (m, 1H); 5.57-5.67 ppm (m, 1H). ¹³C{¹H}-NMR (100.6 MHz, CDCl₃): δ=12.5 (s); 30.3 (s); 62.0 (s); 126.0 (s); 126.5 ppm (s); signals of the by-product ((E)-isomer): δ=17.4; 35.7; 61.9; 127.2; 127.5 ppm.

1.2 (Z)-Pent-3-enoic acid ((Z)-3)

CH₃CN (50 ml) and (Z)-pent-3-en-1-ol (2.36 ml, 2.0 g, 23.22 mmol, 1.0 eq.) were added in succession to a solution of Na₂Cr₂O₂ (69.2 mg, 0.23 mmol, 0.01 eq.), 65% strength nitric acid (450 mg, 4.64 mmol, 0.2 eq.) and NaIO₄ (10.93 g, 51.1 mmol, 2.2 eq.) in H₂O (25 ml) at 0° C. The reaction mixture was stirred at 0° C. for 8 hours and overnight at 10° C. The conversion according to NMR was 98%. Inorganic salts were filtered off and washed with diethyl ether. After separation of the phases, the aqueous phase was extracted with ether (3×100 ml). The organic phases were combined, dried over Na₂SO₄ and freed of the solvent under reduced pressure. The residue (2.04 g) was fractionally distilled (100° C./20 mbar). (Z)-Pent-3-enoic acid was obtained as a colorless liquid in an amount of 1.83 g (yield: 79%). According to ¹H-NMR analysis, the content of (Z)-isomer was 95%.

¹H-NMR (400.1 MHz, CDCl₃): δ=1.65 (ddt, J=6.8; 1.8; 1.8 Hz, 3H); 3.13-3.16 (dm, J=7.2 Hz, 2H); 5.53-5.74 (m, 2H); 10.8 ppm (bs, 1H). ¹³C{¹H}-NMR (100.6 MHz, CDCl₃): δ=13.0 (s); 32.5 (s); 121.0 (s); 128.2 (s); 178.7 ppm (s); signals of the by-product ((E)-isomer): δ=18.0; 37.9; 122.0; 130.2; 179.0 ppm.

2. Preparation of 2-Vinylhept-6-enoic acid (4)
2.1. Pent-4-enyl p-toluenesulfonate

p-Toluenesulfonyl chloride (22.15 g, 116 mmol, 1.5 eq.) was added in small portions to a solution of pent-4-en-1-ol (6.67 g, 77.5 mmol, 1 eq.) and pyridine (abs., 12.53 ml, 12.25 g, 154.9 mmol, 2 eq.) in CH₂Cl₂ (80 ml) at 0° C. The reaction mixture was stirred at 0° C. for 3 hours. After addition of water (60 ml), the mixture was extracted with diethyl ether (125 ml). The organic phase was washed in succession with aqueous hydrochloric acid (2 M), an aqueous Na₂CO₃ solution (5%) and water, dried over MgSO₄ and freed of the solvent under reduced pressure. The residue was fractionated by column chromatography (petroleum ether/diethyl ether, 8:1). Pent-4-enyl p-toluenesulfonate was obtained as a colorless oil in an amount of 17.7 g (yield: 95%).

¹H NMR (400.1 MHz, CDCl₃): δ=1.71-1.78 (m, 2H); 2.05-2.11 (m, 2H); 2.45 (s); 4.04 (t, J=6.4 Hz, 2H); 4.93-4.98 (m, 2H); 5.64-5.74 (m, 1H); 7.35 (dm, J=8.3 Hz, 2H); 7.79 ppm (dm, J=8.3 Hz, 2H). ¹³C{¹H}-NMR (100.6 MHz, CDCl₃): δ=21.6; 28.0; 29.4; 69.8; 115.8; 127.9; 129.8; 133.2; 136.6; 144.7 ppm.

2.2 2-Vinylhept-6-enoic acid (4)

n-Butyllithium (2.5 M in hexane, 39.9 ml, 99.84 mmol, 2.4 eq.) was added slowly to a solution of diethylamine (7.3 g, 10.28 ml, 99.84 mmol, 2.4 eq.) in tetrahydrofuran (THF, 50 ml, abs.) at −78° C. under argon. The reaction mixture was stirred at 0° C. for 0.5 hour and subsequently cooled back down to −78° C. At this temperature, a solution of (E)-but-2-enoic acid (4.3 g, 49.92 mmol, 1.2 eq.) in THF (abs., 50 ml) was added to this reaction mixture over a period of 15 minutes. The reaction mixture was subsequently stirred at 0° C. for 1 hour and cooled back down to −78° C. A solution of pent-4-enyl para-toluenesulfonate (10 g, 41.6 mmol, 1 eq.) in THF (50 ml, abs.) was added to this reaction mixture at −78° C. over a period of 1 hour by means of a syringe pump. The reaction mixture was warmed to −20° C. over a period of 1 hour and stirred at this temperature for a further 16 hours. H₂O (300 ml) was subsequently added and the mixture was washed with diethyl ether (3×200 ml). The aqueous phase was acidified with phosphoric acid (85%) while cooling in ice and subsequently extracted with ethyl acetate (3×250 ml). The organic phases were combined, dried over MgSO₄ and freed of the solvent under reduced pressure. The residue was fractionally distilled (80° C./0.4 mbar). 2-Vinylhept-6-enoic acid was obtained as a colorless oil in an amount of 5.2 g (yield: 81%).

¹H-NMR (400.1 MHz, CDCl₃): δ=1.31-1.51 (m, 2H); 1.51-1.63 (m, 1H); 1.75-1.85 (m, 1H); 2.07 (dt, J=7.0; 7.0 Hz, 2H); 3.02 (dt, J=7.7; 7.7 Hz, 1H); 4.93-5.04 (m, 2H); 5.14-5.25 (m, 2H); 5.72-5.86 (m, 2H); 11.73 ppm (bs, 1H). ¹³C{¹H}-NMR (100.6 MHz, CDCl₃): δ=26.2 (s); 31.4 (s); 33.4 (s); 50.0 (s); 114.9 (s); 117.8 (s); 135.4 (s); 138.2 (s); 181.2 ppm (s). MS (CI(NH₃)): [m/e]=109.1 (13%), 172.1 (100%, [M+NH₃+H]⁺). Elemental analysis [%]: calc.: C, 70.1; H, 9.15; found: C, 69.8; H, 9.04.

### IV. General Hydroformylation Method

The compound to be reacted (substrate) is added to a solution of suspension of [Rh(CO)₂acac], the appropriate ligand, 1,3,5-trimethoxybenzene (as internal standard) in a solvent in a Schlenk flask. The reaction mixture is stirred under argon for 5 minutes. The reaction mixture is transferred by means of a syringe under an argon atmosphere to an autoclave. The autoclave is flushed three times with the synthesis gas (CO/H₂, 1:1).

The hydroformylation reactions are carried out in

(A) an Argonaut Endeavour® reactor system comprising eight parallel, mechanically stirred pressure reactors provided with independent temperature and pressure control. The progress of the reaction was determined by evaluating the consumption of synthesis gas;

(B) a Premex Medimex stainless steel autoclave (100 ml) provided with a magnetic stirrer. The autoclave is equipped with a glass liner and a sampling facility. For kinetic studies, the autoclave is thermostated and samples are taken and examined by means of NMR analysis.

The reactions are (if appropriate) interrupted by cooling of the system, venting and flushing of the reactor with argon. Samples are examined by NMR analysis of the crude reaction mixtures in CDCl₃ and/or by NMR analysis of the samples after removal of the solvent.

### V. Hydroformylation Examples to Examine Regioselectivity

### 1. Hydroformylation of Vinylacetic Acid (5):

Experimental conditions: reactor: autoclave (A); molar ratio of [Rh(CO)₂acae]:ligand:(5):standard=1:10:200:100; solvent: THF (2 ml); starting concentration of the substrate (3): c₀(3)=0.2 M; synthesis gas: CO/H₂ (1:1); reaction pressure: 10 bar; reaction temperature: 40° C.; reaction time: 4 h.

### Main Products of the Hydroformylation:

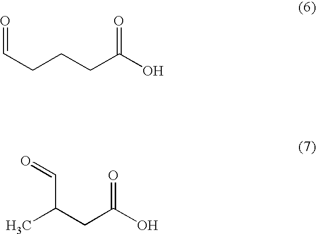

The turnover frequency (TOF; mol(aldehyde)/mol(catalyst) h⁻¹) was determined from the consumption of synthesis gas. After removal of the solvent under reduced pressure (150 mbar) and addition of triethylamine (100 μl), the conversion (in %) and the regioselectivity of the reaction (molar ratio of (6)/(7)) was determined by integration of the characteristic signals of the reaction products formed in the ¹H-NMR spectrum of the reaction mixture formed. Each experiment was repeated at least twice. By-products of this reaction were observed in an amount of <5% in all experiments.

Ligands which are not according to the invention used:

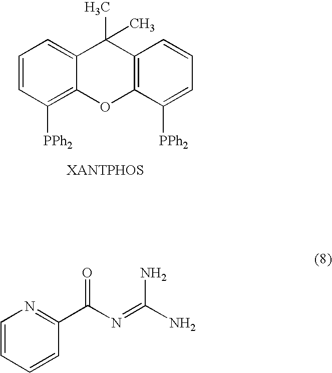

TABLE 1

| | | | Conversion | Regioselectivity |
|---|---|---|---|---|
| Example | Ligand | TOF [h⁻¹] | [%] | (6)/(7) |
| 1.1 | (1) | 250 | 100 | 23 |
| 1.2 | (2) | 60 | 96 | 4.8 |
| 1.3 (CE) | — | 16 | 25 | 0.58 |
| 1.4 (CE) | PPh₃ | 30 | 53 | 1.3 |
| 1.5 (CE) | XANTPHOS | 3 | 5 | >20^{[a]} |
| 1.6 (CE) | XANTPHOS, | 50 | ca. 85 (100) | 15.5 |
| | 80° C.^{[b]}, 4 h | | | |
| | (20 h)^{[c]} | | | |
| 1.7 (CE) | PPh₃/(8), (1:1) | 12 | 20 | 1.5 |

(CE) = Comparative Example (not according to the invention)
^{[a]}Owing to the low conversion, the regioselectivity could not be determined with sufficient accuracy
^{[b]}Reaction temperature deviating from basic method
^{[c]}Reaction time deviating from basic method

### 1.8 Preparation of 5-Oxopentanoic Acid (6) by Hydroformylation of Vinylacetic Acid (5)

Experimental conditions: reactor: autoclave (B); molar ratio of [Rh(CO)₂acac]:(1):(5)=1:20:200; solvent: THF (5 ml); starting concentration of the substrate (5): c₀(5)=0.39 M; synthesis gas: CO/H₂ (1:1); reaction pressure: 4 bar; reaction temperature: room temperature; reaction time: 20 h.

After removal of the solvent under reduced pressure, the residue was taken up in CH₂Cl₂, applied to a short silica gel column and eluted with diethyl ether. 5-Oxopentanoic acid (6) was obtained as a colorless liquid in an amount of 215.5 mg (yield: 96%). According to NMR analysis, the isolated product comprises 1.7 mol % of 3-methyl-4-oxobutyric acid (7) as further component.

¹H-NMR (400.1 MHz, CDCl₃): δ=1.96 (pseudo-q, J=7.2; 7.2 Hz, 2H); 2.44 (t, J=7.2 Hz, 2H); 2.56 (dt, J=1.3; 7.2 Hz, 2H); 9.77 (bs, 1H); 9.85 ppm (bs, 1H). ¹³C{¹H}-NMR (100.6 MHz, CDCl₃): δ=17.0 (s); 32.9 (s); 42.7 (s); 178.9 (s); 201.3 (bs). MS (CI(NH₃): [m/e]=133.9 (100%) [M+NH₃+H]⁺). Elemental analysis [%]: calc.: C, 51.72; H, 6.94; found: C, 51.56; H, 6.71.

2. Hydroformylation of pent-4-enoic Acid (9)

Reaction conditions: reactor: autoclave (A); molar ratio of [Rh(CO)₂acac]:(1):(9):standard=1:10:200:100; solvent: THF (2 ml); starting concentration of the substrate (9): c₀(9)=0.2 M; synthesis gas: CO/H₂ (1:1); reaction pressure: 10 bar; reaction temperature: 40° C.; reaction time: 4 h.

### Possible Products of the Hydroformylation:

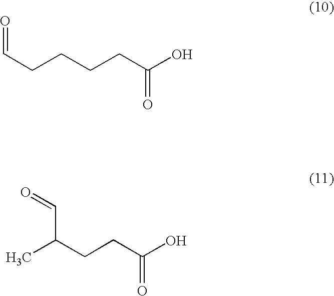

The turnover frequency (TOF; mol(aldehyde)/mol(catalyst) h⁻¹) was determined from the consumption of synthesis gas. After removal of the solvent under reduced pressure (150 mbar), the conversion (in %) and the regioselectivity of the reaction (molar ratio of (10)/(11)) was determined by integration of the characteristic signals of the reaction products formed in the ¹H-NMR spectrum of the reaction mixture formed. Each experiment was repeated at least twice.

By-products of this reaction were observed in an amount of <5% in all experiments.

Result: TOF=49 h⁻¹; conversion: 73%; regioselectivity of the reaction: (10)/(11)=3.6.

4. Hydroformylation of Methyl but-3-enoate (12) (Not According to the Invention)

Reaction conditions: reactor: autoclave (A); molar ratio of [Rh(CO)₂acac]:(1):(12):CH₃COOH:standard=1:10:200:(as shown in Table 2): 100; solvent: THF (2 ml); starting concentration of the substrate (12): c₀(12)=0.2 M; synthesis gas: CO/H₂ (1:1); reaction pressure: 10 bar; reaction temperature: 40° C.; reaction time: 4 h.

### Possible Products of the Hydroformylation:

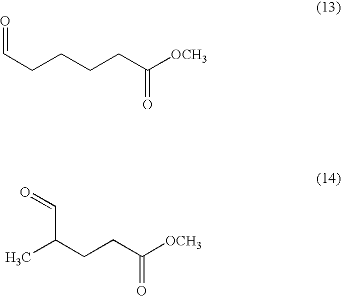

The turnover frequency (TOF; mol(aldehyde)/mol(catalyst) h⁻¹) was determined from the consumption of synthesis gas. The conversion (in %) and the regioselectivity of the reaction (molar ratio of (13)/(14)) was determined by integration of the characteristic signals of the reaction products formed in the ¹H-NMR spectrum of the resulting reaction mixture diluted with CDCl₃. Each experiment was repeated at least twice. By-products of this reaction were observed in an amount of <5% in all experiments.

TABLE 2

| | | | Conversion | Regioselectivity |
|---|---|---|---|---|
| Example | CH₃COOH^{[a]} | TOF [h⁻¹] | [%] | (13)/(14) |
| 4.1^{[b]} | 0 | 29 | 50 | 1.1 |
| 4.2 | 1 | 34 | 58 | 1.4 |

^{[a]}Mol of CH₃COOH per mol of (12)
^{[b]}Suspension (ligand 1 is insoluble in the reaction medium without carboxylic acid).

5. Hydroformylation of (Z)-pent-3-enoic acid ((Z)-3)

Reaction conditions: reactor: autoclave (B); molar ratio of [Rh(CO)₂acac]:ligand:((Z)-3): standard=1:10:50:25; starting concentration of the substrate: ((Z)-3): c₀((Z)-3)=0.2 M; solvent: THF (4 ml); synthesis gas: CO/H₂ (1:1); reaction pressure: 6 bar; reaction temperature: room temperature; reaction time: 68 h.

After removal of the solvent under reduced pressure (150 mbar) and addition of triethylamine (100 μl), the conversion (in %) and the regioselectivity of the reaction (molar ratio of (15)/(16)) were determined by integration of the characteristic signals of the reaction products formed in the ¹H-NMR spectrum of the reaction mixture formed. The results are shown in Table 3.

### Products of the Hydroformylation:

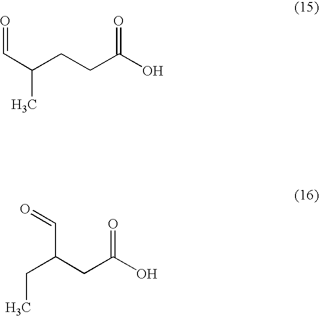

TABLE 3

| | | Composition of the reaction | | |
|---|---|---|---|---|
| | | product [mol-%] | | Regioselectivity |
| Example | Ligand | ((Z)-3):((E)-3):(15):(16) | Conversion [%] | (15):(16) |
| 5.1 | (1) | 10.5:9.0:71.5:6.5 | 80.5 | 11:1 |
| 5.2 (CE) | PPh₃ | 76:3.5:4:7 | 20 | 1:1.7 |

5.3 Preparation of 4-methyl-5-oxopentanoic acid (15) by hydroformylation of (Z)-pent-3-enoic acid ((Z)-3)

Experimental conditions: reactor: autoclave (B); molar ratio of [Rh(CO)₂acac]:(1):((Z)-3)=1:10:50; starting concentration of the substrate ((Z)-3): c₀((Z)-3)=0.2 M; solvent: THF (4 ml); synthesis gas: CO/H₂ (1:1); reaction pressure: 4 bar; reaction temperature: room temperature; reaction time: 68 h.

The reaction mixture obtained was admixed with silica gel (1 g) and freed of the solvent under reduced pressure. The resulting solid was applied to a silica gel column and fractionated by chromatography (eluent: petroleum ether/diethyl ether/acetic acid, 100:50:1). A product mixture of (15) and (16) was obtained as a colorless solid in an amount of 70 mg (yield: 67.2%). The product mixture comprised 92% of 4-methyl-5-oxopentanoic acid (15) and 8% of 3-formylpentanoic acid (16). 7.4 mg (9.2%) of the starting compound and its (E)-isomer were recovered.

Rf (SiO₂, petroleum ether/diethyl ether/acetic acid=100:50:1)=0.12. ¹H-NMR (400.1 MHz, CDCl₃): δ=1.15 (d, J=7.1 Hz, 3H); 1.66-1.75 (m, 1H); 2.02-2.11 (m, 1H); 2.44 (t, J=7.5 Hz, 2H); 2.40-2.50 (m, 1H); 9.62 (bs, 1H); 10.6 ppm (bs, 1H). ¹³C{1H}-NMR (100.6 MHz, CDCl₃): δ=13.2 (s); 24.9 (s); 31.1 (s); 45.2 (s); 179.0 (s); 204.2 ppm (s). MS (CI(NH₃)): [m/e]=113 (100% [M−H₂O+H]⁺), 131 (33% [M+H]⁺), 148 (40% [M+NH₃+H]⁺). Elemental analysis [%]: calc.: C, 55.37; H, 7.74; found: C, 55.29; H, 7.54.

### 6. Hydroformylation of Vinylacetic Acid (5) in the Presence of an Inhibitor

Experimental conditions: reactor: autoclave (A); molar ratio of [Rh(CO)₂acac]:(1):inhibitor:standard=1:10:(as shown in Table 4):200:100; starting concentration of the substrate (5): c₀(5)=0.2 M; solvent: THF (2 ml); synthesis gas: CO/H₂ (1:1); reaction pressure: 10 bar; reaction temperature: 40° C.; reaction time: 4 h.

As regards the further experimental conditions, reference may be made to what has been said under paragraph V.1 in respect of the evaluation of the reaction of vinylacetic acid (5). The results are summarized in Table 4.

TABLE 4

| | Inhibitor | | Conversion | Regioselectivity |
|---|---|---|---|---|
| Example | (CH₃CO₂H)^{[a]} | TOF [h⁻¹] | [%] | (4)/(5) |
| 6.1 | 0 | 250 | 100 | 23 |
| 6.2 | 1 | 195 | 100 | 15 |
| 6.3 | 2 | 107 | 100 | 10 |
| 6.4 | 3 | 96 | 98 | 8 |
| 6.5 | 5 | 92 | 93 | 5 |

^{[a]}mol of CH₃CO₂H based on 1 mol of vinylacetic acid (5).

### VI. Hydroformylation Examples to Examine Chemoselectivity

### 1. Hydroformylation of Vinylacetic Acid (5) in the Presence of Methyl Vinyl Acetate (17)

Reaction conditions: reactor: autoclave (B); molar ratio of [Rh(CO)₂acac]:ligand:(5):(17):standard=1:20:200:200:25; starting concentration of the substrates (5) and (17): c₀(5)=c₀(17)=0.13 M; solvent: THF (6 ml); synthesis gas: CO/H₂ (1:1); reaction pressure: 4 bar; reaction temperature: room temperature.

### Products of the Hydroformylation of (17):

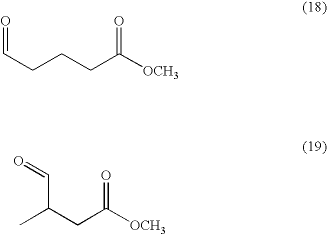

The conversion of (5) and (17) (in %) and the regioselectivity of the reaction of (17) ((18)/(19)) were determined by NMR analysis of the crude reaction mixture diluted with CDCl₃. The regioselectivity of the reaction of (5) ((6)/(7)) was determined after removal of the solvent from the reaction mixture. The results are shown in Table 5.

TABLE 5

| | Reaction | Conversion of | | Conversion of | |
|---|---|---|---|---|---|
| Ex. | time (h) | (5) [%] | (6)/(7) | (17) [%] | (18)/(19) |
| 1.1 | 12 | 58 | >50 | 6 | not det. |
| 1.2 | 20 | 100 | >50 | 28 | 2.3 |

2. Hydroformylation of Vinylacetic Acid (5) in the Presence of 1-octene (20)

Reaction conditions: reactor: autoclave (B); molar ratio of [Rh(CO)₂acac]:(1):(5):(20):standard=1:20:200:200:100; starting concentration of the substrates (5) and (20): c₀(5)=c₀(20)=0.13 M; solvent: THF (6 ml); synthesis gas: CO/H₂ (1:1); reaction pressure: 4 bar; reaction temperature: room temperature.

### Products of the Hydroformylation of (20):

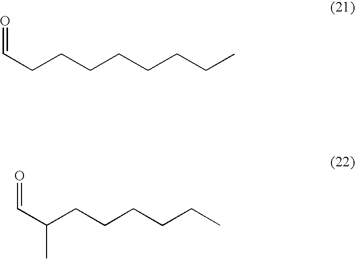

The conversion of (5) and (20) (in %) and the regioselectivity of the reaction of (20) ((21)/(22)) was determined by NMR analysis of the crude reaction mixture diluted with CDCl₃. The regioselectivity of the reaction of (5) ((6)/(7)) was determined after removal of the solvent from the reaction mixture. The results are shown in Table 6.

TABLE 6

| | Reaction | Conversion | | Conversion | |
|---|---|---|---|---|---|
| Example | time [h] | of (5) [%] | (6)/(7) | of (20) [%] | (21)/(22) |
| 2.1 | 6 | 22 | >50 | <3 | — |
| 2.2 | 15.5 | 100 | >50 | 25 | 3 |

3. Hydroformylation of 2-vinylhept-6-enoic Acid (23) (Internal Selectivity)

Experimental conditions: reactor: autoclave (B); molar ratio of [Rh(CO)₂acac]:ligand:(23):standard=1:10:150:50; starting concentration of the substrate (23): c₀(23)=0.2 M; solvent: THF (8 ml); synthesis gas: CO/H₂ (1:1); reaction pressure: 4 bar; reaction temperature: 25° C.

Reaction sites for the Hydroformylation of (23):

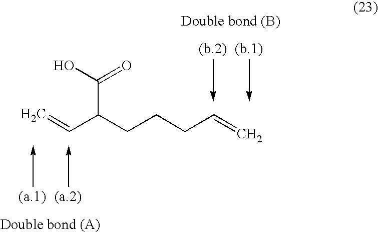

### Double Bond (A)

Samples (0.5 ml) were taken from the hydroformylation reaction at the times shown in Tables 7 and 8. After removal of the solvent from the samples, they were used to determine the selectivity of the hydroformylation reaction in respect of the double bonds ((A)/(B)) and the regioselectivity of the hydroformylation reaction at the respective double bonds ((a.1)/(a.2) or (b.1)/(b.2)) by NMR analysis. The results of the hydroformylation of (23) in the presence of the ligand (1) are shown in Table 7. For comparative purposes, the results of the hydroformylation of (23) in the presence of triphenylphosphine as ligand are shown in Table 8.

TABLE 7
Reaction of (23) in the presence of the ligand (1)

| | | Conversion of (A) [%]; | Conversion of (B) [%]; |
|---|---|---|---|
| | | conversion ratio of | conversion ratio of |
| Example | Time [h] | (a.1)/(a.2) | (b.1)/(b.2) |
| 3.1 | 1 | 6; n.d. | 0; n.d. |
| 3.2 | 2 | 21; n.d. | 1.7; n.d. |
| 3.3 | 3 | 52; n.d. | 3.5; n.d. |
| 3.4 | 4 | 75; (72.7/2.3) = 32 | 6.5; (4.5/1.7) = 2.6 |
| 3.5 | 5 | 88; (85.4/2.6) = 33 | 11.8; (9.3/2.5) = 3.7 |
| 3.6 | 6.25 | 95; (92/3) = 31 | 16.1; (12.3/3.8) = 3.2 |
| 3.7 | 8.5 | 100; (97.1/2.9) = 33 | 24.7; (19.4/5.3) = 3.7 |

From these results, the turnover frequencies in respect of the two double bonds of (23) when using the ligand (1) can be calculated as TOF(A)=46.5 h⁻¹ and TOF(B)=5.3 h⁻¹.

TABLE 8
Reaction of (23) in the presence of PPh₃ as ligand
(not according to the invention)

| | | Conversion of (A) [%]; | Conversion of (B) [%]; |
|---|---|---|---|
| | | conversion ratio of | conversion ratio of |
| Example | Time [h] | (a.1)/(a.2) | (b.1)/(b.2) |
| 3.8 (CE) | 4.7 | 4.5; n.d. | 9; n.d. |
| 3.9 (CE) | 9.5 | 13.5; (9/4.5) = 2.0 | 23.3; (17/6.3) = 2.7 |
| 3.10 (CE) | 22 | 44.3; (29.8/14.5) = 2.1 | 61.9; (46.6/15.3) = 3.0 |
| 3.11 (CE) | 25.3 | 54; (36.5/17.5) = 2.1 | 71.3; (51.9/19.4) = 2.7 |

From these results, the turnover frequencies in respect of the two double bonds of (23) when using triphenylphosphine as ligand can be calculated as TOF(B)=4.6 h⁻¹ and TOF(A)=3.7 h⁻¹.

3.12 Preparation of 2-(3-oxopropyl)hept-6-enoic acid (24) by hydroformylation of 2-vinylhept-6-enoic acid (23)

Experimental conditions: reactor: autoclave (B); molar ratio of [Rh(CO)₂acae]:(1):(23)=1:10:150; starting concentration of the substrate (23): c₀(23)=0.2 M; solvent: THF (8 ml); synthesis gas: CO/H₂ (1:1); reaction pressure: 4 bar; reaction temperature: 25° C.; reaction time: 6.25 h.

The reaction mixture obtained after the reaction was complete was admixed with silica gel (1 g) and freed of the solvent under reduced pressure. The resulting solid was applied to a silica gel column and fractionated by chromatography (eluent: petroleum ether/diethyl ether/acetic acid, 100:50:1). 2-(3-Oxopropyl)hept-6-enoic acid (24) was obtained as a colorless liquid (220 mg, yield: 74.6%). In addition, 17.6 mg (7.1%) of the starting compound (23) were recovered.

Rf (SiO₂, petroleum ether/diethyl ether/acetic acid=100:50:2)=0.27. ¹H-NMR (400.1 MHz, CDCl₃): δ=1.39-1.57 (m, 3H); 1.64-1.74 (m, 1H); 1.83-1.97 (m, 2H); 2.44 (ddt, 2H, J=7.0; 7.0; 1.3 Hz); 2.38-2.45 (m, 1H); 2.47-2.61 (m, 2H); 4.95-5.04 (m, 2H); 5.73-5.83 (m, 1H); 9.77 (bs, 1H), 11.14 ppm (bs, 1H). ¹³C{¹H}-NMR (100.6 MHz, CDCl₃): δ=23.8 (s); 26.2 (s); 31.4 (s); 33.4 (s); 41.4 (s); 44.3 (s); 114.9 (s); 138.0 (s); 181.8 (s); 201.4 ppm (s). MS (Cl(NH₃)): [m/e]=110.0 (55%), 167.0 (67% [M−H₂O+H]⁺), 185.1 (100% [M+H]⁺), 202.1 (95% [M+NH₃+H]⁺). Elemental analysis [%]: calc.: C, 65.19; H, 8.75; found: C, 65.0; H, 8.69.

### VII. Molecular Modeling

The mutual recognition capability of catalyst and substrate was checked for the catalyst/substrate pair of example V.1.1 (Rh(CO)₂acac/(1)/vinylacetic acid) by molecular modeling (MMFF, Spartan Pro). The data obtained support the experimental findings with regard to the mutual recognition of catalyst and substrate.

1-22. (canceled)

23. A process for the hydroformylation of compounds of the formula (I), 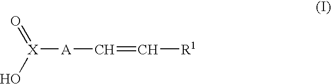
where
X is C, P(R^{x}), P(O—R^{x}), S or S(═O), where R^{x} is H, alkyl, cycloalkyl, heterocycloalkyl, aryl or hetaryl, where alkyl may have 1, 2, 3, 4 or 5 substituents selected from among halogen, cyano, nitro, alkoxy, cycloalkyl, cycloalkoxy, heterocycloalkyl, heterocycloalkoxy, aryl, aryloxy, hetaryl and hetaryloxy and where cycloalkyl, heterocycloalkyl, aryl and hetaryl may have 1, 2, 3, 4 or 5 substituents selected from among alkyl and the substituents mentioned above for alkyl,
A is a divalent bridging group having from 1 to 4 bridging atoms between the flanking bonds and
R¹ is H, alkyl, alkenyl, alkynyl, cycloalkyl, heterocycloalkyl, aryl or hetaryl, where alkyl, alkenyl and alkynyl may have 1, 2, 3, 4 or 5 substituents selected from among halogen, cyano, nitro, alkoxy, cycloalkyl, cycloalkoxy, heterocycloalkyl, heterocycloalkoxy, aryl, aryloxy, hetaryl and hetaryloxy and where cycloalkyl, heterocycloalkyl, aryl and hetaryl may have 1, 2, 3, 4 or 5 substituents selected from among alkyl and the substituents mentioned above for alkyl, alkenyl and alkynyl,
or salts thereof,
in which the compound of the formula (I) is reacted with carbon monoxide and hydrogen in the presence of a catalyst comprising at least one complex of a metal of transition group VIII of the Periodic Table of the Elements with at least one compound of the formula (II), 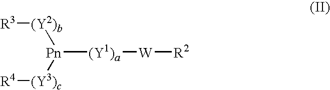
where
Pn is a pnicogen atom;
W is a divalent bridging group having from 1 to 8 bridging atoms between the flanking bonds,
R² is a functional group capable of forming at least one intermolecular, noncovalent bond with the —X(═O)OH group of the compound of the formula (I),
R³ and R⁴ are each, independently of one another, alkyl, cycloalkyl, heterocycloalkyl, aryl or hetaryl, where alkyl may have 1, 2, 3, 4 or 5 substituents selected from among halogen, cyano, nitro, alkoxy, cycloalkyl, cycloalkoxy, heterocycloalkyl, heterocycloalkoxy, aryl, aryloxy, hetaryl and hetaryloxy and where cycloalkyl, heterocycloalkyl, aryl and hetaryl may have 1, 2, 3, 4 or 5 substituents selected from among alkyl and the substituents mentioned above for alkyl; or
together with the pnicogen atom and, if present, the radicals Y² and Y³ form a 5- to 8-membered heterocycle which may additionally be fused with one, two, three or four cycloalkyl, heterocycloalkyl, aryl or hetaryl groups, where the heterocycle and, if present, the fused-on groups each have, independently of one another, 1, 2, 3, 4 or 5 substituents selected from among halogen, cyano, nitro, alkyl, alkoxy, cycloalkyl, cycloalkoxy, heterocycloalkyl, heterocycloalkoxy, aryl, aryloxy, hetaryl and hetaryloxy,
a, b and c are each, independently of one another, 0 or 1 and
Y¹, Y² and Y³ are each, independently of one another, O, S, NR^{a} or SiR^{b}R^{c}, where R^{a}, R^{b} and R^{c} are each, independently of one another, hydrogen, alkyl, cycloalkyl, heterocycloalkyl, aryl or hetaryl, where alkyl may have 1, 2, 3, 4 or 5 substituents selected from among halogen, cyano, nitro, alkoxy, cycloalkyl, cycloalkoxy, heterocycloalkyl, heterocycloalkoxy, aryl, aryloxy, hetaryl and hetaryloxy and where cycloalkyl, heterocycloalkyl, aryl and hetaryl may, if appropriate, have 1, 2, 3, 4 or 5 substituents selected from among alkyl and the substituents mentioned above for alkyl.

24. The process of claim 23, wherein the catalyst is capable of forming, owing to the presence of the group R², an aggregate with the compound of the formula (I), with the C—C double bond of the compound of the formula (I) being capable of interacting with the complexed metal of transition group VIII.

25. The process of claim 23, wherein X in the compounds of the formula (I) is C, S(═O) or P(O—R^{x}), where R^{x} is H or in each case optionally substituted alkyl, cycloalkyl or aryl.

26. The process of claim 25, wherein X is C.

27. The process of claim 23, wherein the compound of the formula (I) is selected from among compounds of the formula (I.a) 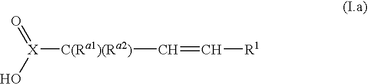
where
X is C, P(R^{x}), P(O—R^{x}), S, S(═O), where R^{x} is H or in each case optionally substituted alkyl, cycloalkyl or aryl,
R^{a1} and R^{a2} are each, independently of one another, H or C₁-C₄-alkyl and
R¹ is H, alkyl, alkenyl, alkynyl, cycloalkyl, heterocycloalkyl, aryl or hetaryl.

28. The process of claim 23, wherein the metal of transition group VIII of the Periodic Table of the Elements is selected from among Co, Ru, Rh, Ir, Pd and Pt.

29. The process of claim 28, wherein the metal of transition group VIII of the Periodic Table is Rh.

30. The process of claim 23, wherein Pn in the compounds of the formula (II) is phosphorus.

31. The process of claim 23, wherein the radical R² in the compound of the formula (II) comprises at least one NH group.

32. The process of claim 31, wherein R² is selected from among —NHR^{w}, ═NH, —C(═O)NHR^{w}, —C(═S)NHR^{w}, —C(═NR^{y})NHR^{w}, —O—C(═O)NHR^{w}, —O—C(═S)NHR^{w}, —O—C(═NR^{y})NHR^{w}, —N(R^{z})—C(═O)NHR^{w}, —N(R^{z})—C(═S)NHR^{w} and —N(R^{z})—C(═NR^{y})NHR^{w}, where R^{w}, R^{y} and R^{z} are each, independently of one another, H, alkyl, cycloalkyl, aryl or hetaryl or in each case together with a further substituent of the compound of the formula (II) are part of a 4- to 8-membered ring system.

33. The process of claim 32, wherein R² is —NH—C(═NH)NHR^{w}, where R^{w} is H, alkyl, cycloalkyl, aryl or hetaryl.

34. The process of claim 23, wherein R³ and R⁴ are selected from among in each case optionally substituted phenyl, pyridyl or cyclohexyl.

35. The process of claim 23, wherein a, b and c are each 0.

36. The process of claim 23, wherein the compound of the formula (II) is selected from among compounds of the formula (II.a), 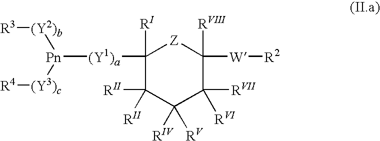
where
a, b, c, Pn, R², R³, R⁴, Y¹, Y² and Y³ have one of the meanings given in claim 23,
W′ is a divalent bridging group having from 1 to 5 bridging atoms between the flanking bonds,
Z is O, S, S(═O), S(═O)₂, N(R^{IX}) or C(R^{IX})(R^{X}) and
R^{I}, R^{II}, R^{III}, R^{IV}, R^{V}, R^{VI}, R^{VII}, R^{VIII}, R^{IX} and R^{x} are each, independently of one another, H, halogen, nitro, cyano, amino, alkyl, alkoxy, alkylamino, dialkylamino, cycloalkyl, heterocycloalkyl, aryl or hetaryl,
or two radicals R^{I}, R^{II}, R^{IV}, R^{VIII} and R^{IX} bound to adjacent ring atoms together represent the second part of a double bond between the adjacent ring atoms, where the six-membered ring can have up to three noncumulated double bonds.

37. The process of claim 36, wherein W′ in the compound of the formula (II.a) is C(═O).

38. The process of claim 36, wherein R² is —NH—C(═NH)NHR^{w}, where R^{w} is H, alkyl, cycloalkyl, aryl or hetaryl.

39. The process of claim 36, wherein the radicals R^{I} together with R^{II}, R^{IV} together with R^{VI} and R^{VIII} together with R^{IX} in the compound of the formula (II.a) in each case represent the second part of a double bond between the adjacent ring atoms.

40. A compound of the formula (II.a) as defined in claim 36.

41. The compound of claim 40 selected from among the compounds of the formulae (1) and (2)

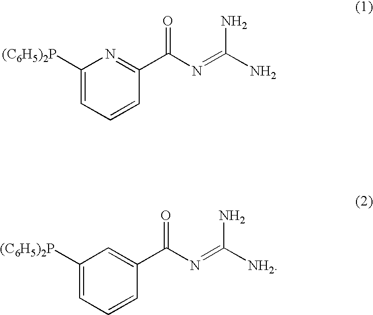

42. A catalyst comprising at least one complex of a metal of transition group VIII of the Periodic Table of the Elements with at least one compound of the formula (II.a) as defined in claim 40.

43. The catalyst of claim 42, wherein the metal of transition group VIII of the Periodic Table of the Elements is selected from among Co, Ru, Rh, Ir, Pd and Pt.
